(19) United States
(12) Patent Application Publication
(10) Pub. No.: US 2009/0239298 A1
(43) Pub. Date: Sep. 24, 2009

(54) Methods of generating embryoid bodies using three dimensional scaffolds

(75) Inventors: Sharon Gerecht, Baltimore, MD (US); Smadar Cohen, Beer-Sheva (IL); Joseph Itskovitz-Eldor, Haifa (IL)

Correspondence Address: MARTIN D. MOYNIHAN d/b/a PRTSI, INC., P.O. BOX 16446, ARLINGTON, VA, 22215, US

(73) Assignees: Technion Research & Development Foundation Ltd., Haifa (IL); Ben Gurion University of the Negev Research and Development Authority, Beer-Sheva (IL)

(21) Appl. No.: 11/661,128

(22) PCT Filed: Aug. 10, 2005
(86) PCT No.: PCT/IL05/00865

§ 371 Date: Mar. 13, 2009

Related U.S. Application Data
(60) Provisional application No. 60/604,002, filed on Aug. 25, 2004.

(51) Int. Cl. C12N 5/02; C12N 5/08

(52) U.S. Cl. 435/366; 435/377; 435/396; 435/395

(57) ABSTRACT

A method of generating embryoid bodies is provided. The method comprising culturing undifferentiated embryonic stem cells on a three dimensional scaffold under conditions suitable for formation of embryoid bodies, thereby generating the embryoid bodies.

### FIELD AND BACKGROUND OF THE INVENTION

The present invention relates to methods of generating embryoid bodies using three-dimensional scaffolds and, more particularly, to methods of generating differentiated cells from such embryoid bodies.

Human embryonic stem cells (hESCs) are proliferative, undifferentiated stem cells capable of being maintained in an undifferentiated state while preserving their pluripotent capacity. Upon removal from cultures, ESCs can spontaneously differentiate into three-dimensional cell aggregates, which, overtime, increase in cell number and complexity and form embryoid bodies (EBs, Thomson et al., 1998). EBs are three-dimensional structures comprised of a population of ESCs and/or embryonic germ cells (EGCs), which have undergone differentiation. EB formation is triggered by the removal of differentiation blocking factors from ES cell cultures. In the first step of EB formation, ESCs proliferate into small masses of cells, which then proceed with differentiation. In the first phase of differentiation, following 1-4 days of ESC culture, a layer of endodermal cells is formed on the outer layer of the small mass, resulting in the formation of “simple EBs”. In the second phase, following 3-20 days post-differentiation, “complex EBs” are formed which are featured by extensive differentiation of ectodermal and mesodermal cells and derivative tissues.

Stem-cell-derived-differentiated cells of specific lineages are of increasing importance for various therapeutic and tissue engineering applications. However, for both tissue regeneration and cell-replacement applications there is a need to develop methods of efficiently producing large quantities of EBs-derived-differentiated cells.

Prior art methods of generating EBs involve the initial aggregation of ESCs into spheroid, three-dimensional structures. Thus, when undifferentiated ESCs are removed from their feeder layers and transferred to liquid media using non-adherent tissue culture plates large aggregates of EBs are formed (Itskovitz-Eldor et al., 2000). However, the extent of EB aggregation should be carefully monitored and controlled since large agglomerated EBs are often accompanied with extensive cell death and necrosis due to mass transport limitations (Dang et al., 2002).

To overcome these limitations, methods of controlled agglomeration of EBs have been developed. These include the hanging drop method in which the ESCs are aggregated in hanging drops for two days prior to their transfer to liquid cultures. Another method utilizes semi-solid methylcellulose cultures in order to control EB's agglomeration (as disclosed by Dang et al., 2002 and in U.S. Pat. Appl. No. 20030119107). However, although adequate for small-scale laboratory purposes these systems are not amenable to large-scale clinical production due to the inability to control the extracellular components, which may affect EB purity and reproducibility, as well as the cell types which can be readily generated therefrom [Maltepe, E. et al., (1997). Abnormal angiogenesis and responses to glucose and oxygen deprivation in mice lacking the protein ARNT. Nature, 386: 403-7; Soria, B. (2001). In vitro differentiation of pancreatic beta-cells. Differentiation 68: 205-19; Zandstra, P. W. and Nagy, A. (2001). Stem cell bioengineering. Annu Rev Biomed Eng 3:275-305].

There is thus a widely recognized need for, and it would be highly advantageous to have, a method of generating hEBs devoid of the above described limitations.

### SUMMARY OF THE INVENTION

According to one aspect of the present invention there is provided a method of generating embryoid bodies comprising culturing undifferentiated embryonic stem cells on a three dimensional scaffold under conditions suitable for formation of embryoid bodies, thereby generating the embryoid bodies.

According to another aspect of the present invention there is provided a method of generating expanded and/or differentiated cells from embryonic stem cells comprising: (a) culturing undifferentiated embryonic stem cells on a three dimensional scaffold under conditions suitable for formation of embryoid bodies to thereby obtain embryoid bodies; (b) isolating lineage specific cells from the embryoid bodies; and (c) culturing the lineage specific cells under culturing conditions selected suitable for the expansion and/or differentiation of the lineage specific cells to thereby obtain expanded and/or differentiated lineage-specific cells.

According to yet another aspect of the present invention there is provided a method of treating a disorder requiring cell replacement therapy comprising: (a) culturing undifferentiated embryonic stem cells on a three dimensional scaffold under conditions suitable for formation of embryoid bodies to thereby obtain embryoid bodies; (b) isolating lineage specific cells from the embryoid bodies; (c) culturing the lineage specific cells under culturing conditions selected suitable for the expansion and/or differentiation of the lineage specific cells to thereby obtain expanded and/or differentiated lineage-specific cells, and (d) administering cells of the expanded and/or differentiated lineage-specific cells to an individual in need thereof thereby treating the disorder requiring cell replacement therapy.

According to further features in preferred embodiments of the invention described below, the three dimensional scaffold is a porous scaffold.

According to still further features in the described preferred embodiments the porous scaffold is an alginate scaffold.

According to still further features in the described preferred embodiments the alginate scaffold is an LF120 alginate scaffold or an LF5/60 alginate scaffold.

According to still further features in the described preferred embodiments the porous scaffold is composed of a synthetic polymer.

According to still further features in the described preferred embodiments the porous scaffold is composed of a natural polymer.

According to still further features in the described preferred embodiments the synthetic polymer is selected from the group consisting of a poly(hydroxy) acid, polyanhydride, poly(ortho)ester and polyurethane.

According to still further features in the described preferred embodiments the poly(hydroxy) acid is selected from the group consisting of PLA, PLGA, PGA and PEG containing co-polymers thereof.

According to still further features in the described preferred embodiments the natural polymer is selected from the group consisting of a polysaccharide and a polypeptide.

According to still further features in the described preferred embodiments the polysaccharide is selected from the group consisting of alginate, chitosan and hyaluronic acid.

According to still further features in the described preferred embodiments an average pore size of the porous scaffold is in a range between 10 to 900 μm in diameter.

According to still further features in the described preferred embodiments an average distance between pores of the porous scaffold is in a range between 5 to 500 μm.

According to still further features in the described preferred embodiments an average porosity of the porous scaffold is at least 70%.

According to still further features in the described preferred embodiments the culturing is effected over a period of 1-35 days.

According to still further features in the described preferred embodiments the culturing is effected for 30 days.

According to still further features in the described preferred embodiments at least 90% of the embryoid bodies are within a diameter size range of 400-800 μm.

According to still further features in the described preferred embodiments at least 85% of the embryoid bodies are devoid of necrotic centers.

According to still further features in the described preferred embodiments conditions suitable for formation of embryoid bodies include a culture medium containing serum.

According to still further features in the described preferred embodiments the culture medium includes 80% KO-DMEM, 20% serum, 0.5% Penicillin-Streptomycin, 1 mM L-glutamine, 0.1 mM β-mercaptoethanol and 1% non-essential amino acid stock.

According to still further features in the described preferred embodiments isolating lineage specific cells is effected by sorting of cells contained within the embryoid bodies via fluorescence activated cell sorter.

According to still further features in the described preferred embodiments isolating lineage specific cells is effected by a mechanical separation of cells, tissues and/or tissue-like structures contained within the embryoid bodies.

According to still further features in the described preferred embodiments isolating lineage specific cells is effected by subjecting the embryoid bodies to differentiation factors to thereby induce differentiation of the embryoid bodies into lineage specific differentiated cells.

The present invention successfully addresses the shortcomings of the presently known configurations by providing methods of generating embryoid bodies using three dimensional scaffolds.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are scanning electron micrographs of hESC-seeded LF120 alginate scaffolds following one month of culturing. FIG. 1a shows a scaffold seeded with low hESC concentration, demonstrating the porous structure of the alginate scaffold.

FIG. 1b is a low magnification picture of a scaffold seeded with high hESC concentration, demonstrating homogenous cell distribution throughout the scaffold.

FIGS. 2a-b are light microscope micrographs showing hEB formation within LF5/60 alginate scaffold pores. FIG. 2a shows 4-day-old hEBs formed within the LF5/60 scaffold pores exhibiting moderate distribution on the entire scaffold. FIG. 2b shows 20-day-old hEBs rupturing the LF 5/60 scaffold structure and burst out to the external medium. Bar=100 μm.

FIGS. 2c-d are scanning electron micrographs showing hEB formation within LF120 alginate scaffold pores following one month in culture. FIG. 2c shows hEB development mainly within the confining space of the scaffold pores, and a frequent burst out of the scaffold pores (FIG. 2d).

FIG. 3 is a graph depicting cell proliferation in scaffold-borne hEBs compared to hEBs formed in 2D conditions, as determined by an XTT assay.

FIGS. 4a-f are photomicrographs showing morphology and differentiation of scaffold-borne hEBs. FIG. 4a shows relatively round hEBs formed in alginate scaffolds (i) and in bioreactors (ii). Highly aggregated EBs were formed in 2D static conditions (iii). Different structures within the scaffold-borne hEBs were observed including epithelial sheets (FIG. 4b); voids (FIG. 4c, dashed arrows); and connective mesodermal differentiation (arrowhead) near an endodermal-like tube (FIG. 4d, solid arrow). Differentiated cells included representatives of the ectodermal germ layer (FIG. 4e) such as neuronal-like cells stained positive for nestin as shown in low (i) and high (ii) magnifications, and the endodermal germ layer (FIG. 4f) where sheets of epithelial cells were stained positive for AFP. Scaffolds are marked with an asterisk. Bar=100 μm.

FIGS. 5a-g are photomicrographs depicting vasculogenesis in scaffold-borne hEBs with H&E staining (FIGS. 5a-b) and immunolabeling (FIGS. 5c-g). FIG. 5a shows different types of voids surrounded by cells resembling elongated endothelial morphology (arrowheads). FIG. 5b shows the same structure of FIG. 5a in the presence of the remaining scaffold structure designated with an asterisk. FIG. 5c shows CD34+ cells-surrounded voids (arrowheads). FIGS. 5d-e show higher magnifications of CD34+ cells demonstrating the formation of complex vasculature arrangements along the scaffold walls (dashed arrows). Representative photos of CD34+ immuno-labeled vasculature formed within static and bioreactor borne-hEBs are shown in FIGS. 5f and g, respectively. Bar=100 μm.

### DESCRIPTION OF THE PREFERRED EMBODIMENTS

The present invention is of methods of generating embryoid bodies using three dimensional scaffolds, which can be used for isolating multipotent lineage specific cells.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Human embryonic stem cells (hESCs) are important as a potential source for all types of differentiated cells which can be used in various therapeutic and tissue engineering applications.

Although there are numerous approaches for generating stem cell-derived-differentiated cells, approaches which employ an embryoid body (EB) forming step are presently preferred since they imitate embryogenesis.

EBs are formed following the removal of ESCs from feeder layer-, or matrix-based cultures into suspension cultures. The first and most critical step in the development of EB is the formation of ESC aggregates. The extent of aggregation should be carefully monitored and controlled since large agglomerated EBs are often characterized by extensive cell death and necrosis due to mass transport limitations [Dang et al. (2002). Biotechnol. Bioeng. 78:442-453].

The present inventors have previously demonstrated the large-scale formation of hEBs in a dynamic culture using a rotating cell culture system [see PCT Pat. Appl. No. IL 03/01017 and Gerecht-Nir (2004) Biotechnol. Bioeng. 86: 493-502].

While reducing the present invention to practice the present inventors have uncovered that EBs can be efficiently formed on three dimensional (3D) porous scaffolds and thus can be used for large-scale production of lineage-specific differentiated cells.

As is further illustrated in the Examples section which follows, culturing undifferentiated human stem cells on three dimensional (3D) porous alginate scaffolds resulted in the efficient formation of hEBs (see Examples 1 and 2). Scaffold-borne EBs were of high quality, essentially devoid of necrotic centers, exhibiting high proliferation rate and differentiation to all three germ layers, while exhibiting minimal agglomeration (see Examples 3-5).

It will be appreciated that although similar 3D culturing conditions were previously shown to induce stem cell differentiation, generation of EBs on porous scaffold has not been described or suggest in the prior art. For example, Chen et al. demonstrated limited stem cell differentiation by culturing rhesus ESCs on porous collagen sponges [(2003) Stem Cells 21(3):281-95]. Similarly, Levenberg et al. cultured differentiated hESCs on porous PLLA/PLGA scaffolds, which developed into organized primitive tissue structures. In sharp contrast to the present invention, EBs were never formed using these prior art culturing conditions nor was such a formation attempted. Furthermore, prior art studies employed cells which were already at least partially differentiated once placed on the 3D scaffold (isolated from 8- to 9-day-old EBs) and only a combination of a porous polymeric scaffold and matrigel or fibronectin coating enabled the formation of tissue structures from these differentiated hESCs [Levenberg (2003) Proc. Natl. Acad. Sci. USA 99:4391-6].

Thus, the present invention shows, for the first time, that the confining environment of 3D scaffolds is suitable for efficient EB formation.

Thus, according to one aspect of the present invention there is provided a method of generating embryoid bodies.

As used herein the phrase “embryoid bodies” refers to aggregates of partially differentiated stem cells, which include representatives of all three germ layers including mesoderm, ectoderm and endoderm.

The method according to this aspect is effected by culturing undifferentiated embryonic stem cells on a three dimensional scaffold under conditions suitable for formation of embryoid bodies, thereby generating the embryoid bodies.

As used herein the phrase “three dimensional scaffold” refers to a supporting framework, which promotes the ingrowth of undifferentiated stem cells, cultured thereon.

The three dimensional scaffold of the present invention can be formed from any material. Preferably such a material is biocompatible (i.e., able to exist and perform in a living tissue or a living system by not being toxic or injurious and not causing immunological rejection) and optionally biodegradable (i.e., capable of being broken down into innocuous products when placed within a living system, such as a cell culture system, or a living organism, such as a human or animal, or when exposed to body fluids), bioerodible (capable of being dissolved or suspended in biological fluids) and/or bioresorbable (i.e., capable of being absorbed by the cells, tissue, or fluid in a living body).

Preferably, the three dimensional scaffold according to this aspect of the present invention is a porous scaffold which is composed of polymers.

As used herein a porous scaffold refers to a scaffold which forms a continuous or discontinuous porous network. The porous scaffolds of this aspect of the present invention can be configured as porous beads, porous sponges, porous foams and porous membranes.

The porous scaffold of this aspect of the present invention can be composed of polymers or polymer fibers, which may be synthetic or natural (see U.S. Pat. No. 6,471,993).

Examples of synthetic polymers which can be used in accordance with the present invention include poly(hydroxy acids) such as poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), polyanhydrides, polyorthoesters, poly(ester amides), polyamides, poly(ester ethers)polycarbonates, polyalkylenes such as polyethylene and polypropylene, polyalkylene glycols such as poly(ethylene glycol) (PEG), polyalkylene oxides such as poly(ethylene oxide) (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and copolymers thereof, polyhydroxyalkanoates, poly(propylene fumarate), polyoxymethylene, and poloxamers.

Such polymers can include one or more photopolymerizable groups. The polymers can also be derivatized. For example, the polymers can have substitutions such as alkyl groups, alkylene groups, or other chemical groups. The polymers can also be hydroxylated oxidized, or modified in some other way familiar to those skilled in the art. Blends and co-polymers of these polymers can also be used.

Preferred biodegradable synthetic polymers include poly(hydroxy acids) such as PLA, PGA, PLGA, and copolymers with PEG; polyanhydrides, poly(ortho)esters, polyurethanes, poly(butyric acid), poly(valeric acid), poly(lactide-co-caprolactone), trimethylene carbonate, and other polymers which are described in U.S. Pat. Nos. 5,654,381; 5,627,233; 5,628,863; 5,567,440; and 5,567,435. Typically, these polymers degrade in vivo by both non-enzymatic and enzymatic hydrolysis, and by surface or bulk erosion.

Examples of natural polymers which can be used in accordance with the present invention include polypeptides and polysaccharides such as alginate, dextran, and celluloses; collagens, including derivatized collagens (e.g., alkylated, hydroxylated, oxidized, or PEG-lated collagens, as well as collagens modified by other alterations routinely made by those skilled in the art); hydrophilic proteins such as albumin; hydrophobic proteins such as protamines, and copolymers and mixtures thereof. Typically, these polymers degrade by enzymatic hydrolysis, by exposure to water in vivo, or by surface or bulk erosion.

It will be appreciated that usage of polymer blends may be advantages since these blends may have improved mechanical strength, and controllable degradation rate. Examples of polymer blends, which may be used, include blends of water insoluble polymers and water-soluble polymers.

According to presently known configurations of this aspect of the present invention, the preferred polymer for generating the three dimensional porous scaffold is alginate. Alginate scaffolds are characterized by a macromolecular structure which resembles the extracellular matrix; a hydrogel nature which allows efficient cell seeding [Shapiro and Cohen (1997); Glicklis (2000); and Leor (2000)]; and porosity which can be controlled during fabrication to yield a sponge-like material having more than 90% porosity.

Methods of generating porous scaffolds are described in [see the Materials and Experimental Procedures section of the Examples section which follows, and U.S. Pat. Nos. 6,471,993 and 6,365,149 and references therein; Hutmacher (2001) Scaffold design and fabrication technologies for engineering tissues—state of the art and future perspectives. Journal of Biomaterials Science—Polymer Edition 12(1) 107-124].

The pore size and density of the porous scaffold is preferably controlled by the polymer chemistry and the synthesis methods.

The porous scaffold of this aspect of the present invention has a pore size in a range between 10-900 μm, preferably between 100-900 μm, more preferably between 400-800 μm, even more preferably between 400-700 μm, yet more preferably between 400-600 μm.

The porous scaffold of this aspect of the present invention is featured by an average distance between the pores in a range between 5-500 μm, preferably between 5-270 μm, even more preferably between 10-270 μm, yet more preferably between 10-150 μm; and an average porosity of at least 70%, preferably at least 80%, more preferably at least 90%, say 95%. Scaffold porosity may be measured as described in U.S. Pat. No. 6,471,993.

Once the porous polymer scaffold is formed, it may be, if needed, shaped by methods known to those of skill in the art for shaping solid objects. For example, scaffolds may be shaped by laser ablation, micromachining, use of a hot wire, and by CAD/CAM (computer aided design/computer aided manufacture) processes.

The three dimensional scaffolds of the present invention may be coated with components of extracellular matrix such as fibronectin, laminin, collagen and/or supplemented with cytokines, growth factors and chemokines.

The three dimensional scaffold of this aspect of the present invention may be placed under static culturing conditions such as in a Petri dish or a flask which are commercially available such as from Nalge Nunc Int. Rochester N.Y., USA.

Alternatively, the three dimensional scaffold of the present invention may be placed in a bioreactor [see PCT Pat. Appl. No. IL 03/01017 and Gerecht-Nir (2004) Supra]. Bioreactors which can be used in accordance with the present invention include, but are not limited to, the rotating cell culture systems (RCCS) developed by NASA, which are described in details in U.S. Pat. Nos. 5,763,279 and 5,437,998. Examples of RCCS include the Slow Turning Lateral Vessel (STLV) and the High Aspect Ratio Vessel (HARV) which are further described in PCT Pat. Appl. No. IL 03/01017.

As mentioned hereinabove, in order to obtain EBs, the method of the present invention utilizes undifferentiated stem cells, which are seeded on (within) the 3D scaffold.

As used herein the phrase “undifferentiated stem cells” refers to pluripotent cells which retain self renewal capability and the developmental potential to differentiate into a wide range of cell lineages including the germ line. In contrast, cells present in formed EBs are considered multipotent since they have partially differentiated to form the three germ layers characteristic of EBs.

ESCs of the present invention can be obtained from the embryonic tissue formed after gestation (e.g., blastocyst), or embryonic germ (EG) cells. Stem cell derivation and preparation is further described hereinbelow. Preferred stem cells of the present invention are human embryonic stem cells.

The ESCs of the present invention can be obtained using well-known cell-culturing methods. For example, human ESCs can be isolated from human blastocysts. Human blastocysts are typically obtained from human in vivo pre-implantation embryos or from in vitro fertilized (IVF) embryos. Alternatively, a single cell human embryo can be expanded to the blastocyst stage. For the isolation of human ESCs, the zona pellucida is removed from the blastocyst and the inner cell mass (ICM) is isolated by immunosurgery, and the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ESCs are then routinely split every 1-2 weeks. For further details on methods of preparation human ESCs see Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; Gardner et al., [Fertil. Steril. 69: 84, 1998].

It will be appreciated that commercially available stem cells can also be used by the present invention. Human ESCs can be purchased from the NIH human embryonic stem cells registry (http://escr.nih.gov). Non-limiting examples of commercially available embryonic stem cell lines are BGO1, BG02, BGO3, BG04, CY12, CY30, CY92, CY10, TE03 and TE32.

ESCs used by the present invention can be also derived from human embryonic germ cells (EGCs). Human EGCs are prepared from the primordial germ cells obtained from human fetuses of about 8-11 weeks of gestation using laboratory techniques well known to the skilled artisan. Briefly, genital ridges are dissociated and cut into small chunks which are thereafter disaggregated into cells by mechanical dissociation. EGCs are then grown in tissue culture flasks with the appropriate medium. The cells are cultured with daily replacement of medium until a cell morphology consistent with EGCs is observed, typically after 7-30 days or 1-4 passages. For additional details on methods of preparation human EGCs see Shamblott et al., (1998) Proc. Natl. Acad. Sci. USA 95: 13726, and U.S. Pat. No. 6,090,622.

Once undifferentiated stem cells are obtained, the cells are cultured (seeded) on the 3D scaffold.

Cell seeding is effected in a manner which enables even distribution of the cells on/within the scaffold. One approach which can be utilized to achieve even distribution is seeding under a centrifugal force (Dar et al, 2002) as is further described in Materials and Experimental Procedures section of the Examples section which follows. Cells are preferably seeded at a concentration which ensures entrapment within the scaffold and maximal formation of EBs (see Example 2 of the Examples section which follows). Preferably seeded are 5×10⁶ cells per cm³ scaffold; even more preferably 2.5×10⁷ cells are seeded per cm³ scaffold; even more preferably −5×10⁷ cells are seeded per cm³ scaffold.

Cells are cultured under conditions which are suitable for the formation of EBs. Such conditions include a suitable culture medium, oxygen and gasses.

The culture medium used by the present invention to induce ESC differentiation is preferably knockout KO-DMEM medium which is a water-based medium that includes salts and essential proteins and is available from Gibco-Invitrogen Corporation products, Grand Island, N.Y., USA.

Preferably, the culture medium includes serum or serum replacement. According to specific embodiments, serum is provided at a concentration of at least 5%, more preferably, at least 15% and most preferably at a concentration of 20%.

To reduce intracellular oxidative reactions, β-mercaptoethanol, an anti-oxidant agent, is preferably added to the culture medium.

In addition, to avoid bacterial contamination during culturing, low concentration of antibiotics such as, Penicillin and Streptomycin are added to the culture medium.

According to presently known preferred embodiments the culture medium of the present invention includes 80% KO-DMEM, 20% serum, 0.5% Penicillin-Streptomycin, 1 mM L-glutamine, 0.1 mM β-mercaptoethanol and 1% non-essential amino acid stock, all of which are available from Gibco-Invitrogen Co.

It will be appreciated that the formation of embryo-like structures including all three embryonal germ layers from ESC aggregates is a time-dependent process which depends upon the ability of ESCs to co-localize, communicate with each other and form three-dimensional structures while differentiating into EBs.

According to specific embodiments of the present invention the culturing period needed for generating EBs varies from 1-35 days, depending on the stage of the EB required (e.g., simple or complex EB). In addition, the culturing period can vary depending on the culture medium and 3D scaffold used. Preferably, culturing of EBs is effected for a time period of 30 days or less.

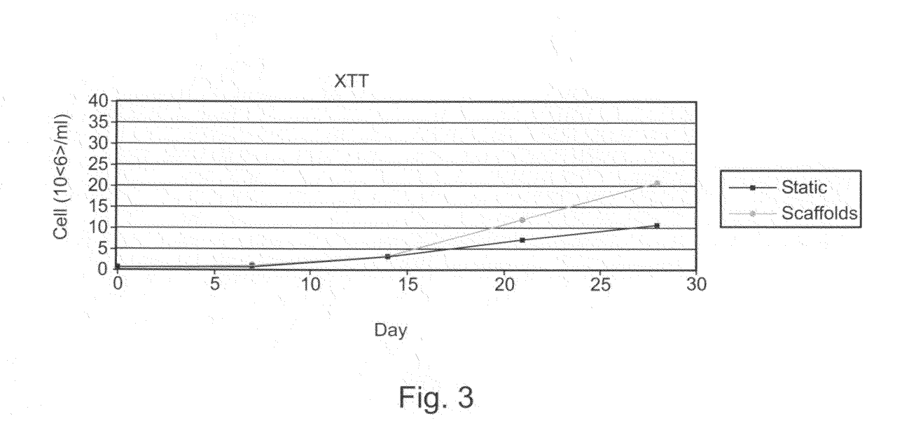

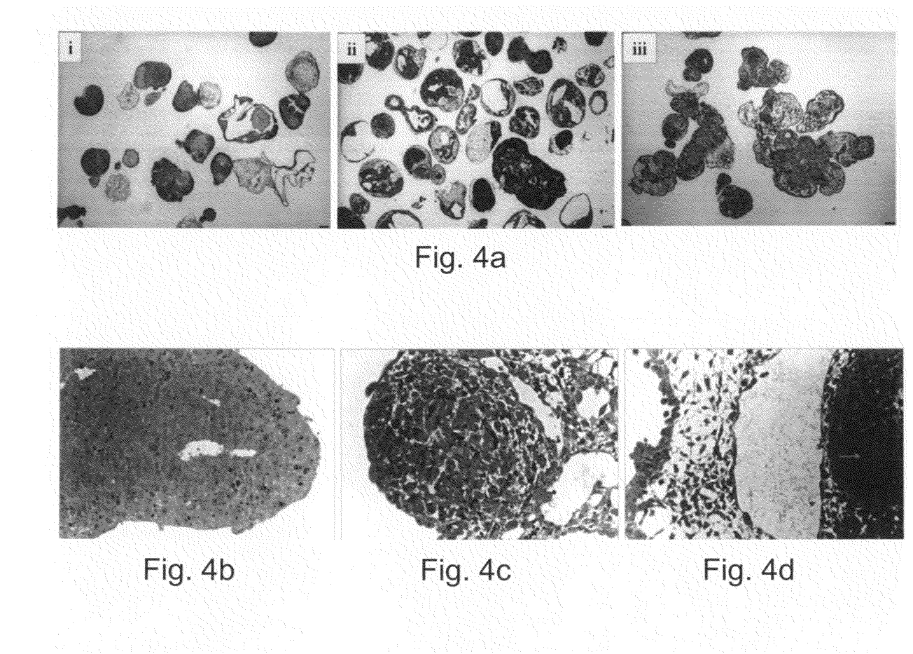

It will be appreciated that EBs can be collected at any time during culturing and examined using an inverted light microscope. Thus, EBs can be examined for their size and shape at any point in the culturing period. Examples of various EB structures are shown in FIGS. 2a-d, 4a-f and 5a-g.

During the culturing step, EBs can be monitored for their viability using methods known in the arts, including, but not limited to, DNA (Brunk, C. F. et al., Analytical Biochemistry 1979, 92: 497-500) and protein (e.g., using the BCA Protein Assay kit, Pierce, Technology Corporation, New York, N.Y., USA) contents, medium metabolite indices, e.g., glucose consumption, lactic acid production, LDH (Cook J. A., and Mitchell J. B. Analytical Biochemistry 1989, 179: 1-7) and medium acidity, as well as by using the XTT method of detecting viable cells [see Example 3 of the Examples section which follows and Roehm, N. et al., An improved colorimetric assay for cell proliferation and viability utilizing the tetrazolium salt XTT J. Immunol. Meth. 142, 257-265 (1991); Scudiero, D. et al., Evaluation of a soluble tetrazolium/formazan assay for cell growth and drug sensitivity in culture using human and other cell lines. Cancer Res. 48, 4827-4833 (1988); Weislow, O. et al., New soluble-formazan assay for HIV-1 cytopathic effects: Application to high-flux screening of synthetic and natural products for AIDS-antiviral activity. J. Natl. Cancer Inst. 81, 577-586 (1989)].

It will be appreciated that viability of cells in culture can also be assessed using various staining methods known in the art. For example, unfixed cells can be stained with the fluorescent dye Ethidium homodimer-1 (excitation, 495 nm; emission, 635 nm) which is detectable in cells with compromised membranes, i.e., dead cells. In this assay, live cells have a green fluorescent cytoplasm but no EthD-1 signal, whereas dead cells lack the green fluorescence and are stained with EthD-1. In addition, the Tunnel assay can be used to label DNA breaks which are characteristics of cells going through apoptosis. Another suitable assay is the live/dead viability/cytotoxicity two-color fluorescence assay, available from Molecular Probes (L-3224, Molecular Probes, Inc., Eugene, Oreg., USA). This assay measures intracellular esterase activity with a cell-permeable substrate (Calcein-AM) which is converted by live cells to a fluorescent derivative (polyanion calcein, excitation, 495 nm; emission, 515 nm) which is thereafter retained by the intact plasma membrane of live cells.

During the culturing period, EBs are further monitored for their differentiation state. Cell differentiation can be determined by their morphology and/or upon examination of cell or tissue-specific markers which are known to be indicative of differentiation. For example, EB-derived-differentiated cells may express the neurofilament 68 KD which is a characteristic marker of the ectoderm cell lineage.

The differentiation level of the EBs can be monitored by following the loss of expression of the embryonic transcription factor Oct-4, and the increased expression level of other markers such as α-fetoprotein, NF-68 kDa, α-cardiac and albumin. Methods of determining the level of gene expression are known in the arts and include, but are not limited to RT-PCR, semi-quantitative RT-PCR, Northern blot, RNA-in situ hybridization and in situ RT-PCR.

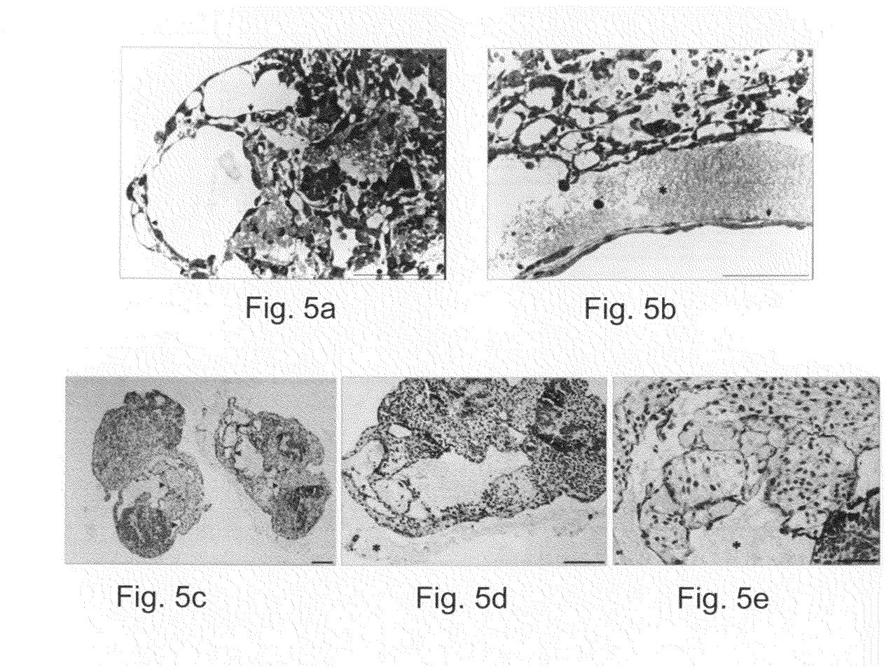

As is shown in Examples 4-5 of the Examples section which follows, EBs formed using the above-described methodology included cells of the three germ layers, ectoderm (see FIG. 4e), endoderm (see FIG. 4f) and mesoderm (see Example 5).

In addition, tissue/cell specific markers can be detected using immunological techniques well known in the art [Thomson J A et al., (1998). Science 282: 1145-7]. Examples include, but are not limited to, flow cytometry for membrane-bound markers, immunohistochemistry for extracellular and intracellular markers and enzymatic immunoassay, for secreted molecular markers.

EBs which are formed using the above-described methodology are preferably a homogeneous culture of small EBs which enable mass transport of nutrients and gasses such as oxygen to and from the cells forming the EBs, thereby preventing cell death and necrosis typical of large EBs (>1000 μm in diameter).

Thus, preferably, at least 90% of the EBs of the present invention have a diameter size range of 250-900 μm, 400-800 μm, even more preferably 400-700 μm and even more preferably 400-600 μm.

The above-described methodology enables generation of a homogeneous population of small-size, viable EBs which are devoid of necrotic centers. The EBs of the present invention are therefore highly useful for large-scale production of EB-derived-lineage specific cells.

Thus, according to another aspect of the present invention there is provided a method of generating expanded and/or differentiated cells from embryonic stem cells.

The method is effected by isolating lineage specific cells from the EBs generated according to the teachings of the present invention and culturing the lineage specific cells under culturing conditions suitable for the expansion and/or differentiation of the lineage specific cells to thereby obtain expanded and/or differentiated lineage-specific cells.

As used herein, the phrase “isolating lineage specific cells” refers to the enrichment of a mixed population of cells in a culture with cells predominantly displaying at least one characteristic associated with a specific lineage phenotype. It will be appreciated that all cell lineages are derived from the three embryonic germ layers. Thus, for example, hepatocytes and pancreatic cells are derived from the embryonic endoderm, osseous, cartilaginous, elastic, fibrous connective tissues, myocytes, myocardial cells, bone marrow cells, vascular cells (namely endothelial and smooth muscle cells), and hematopoietic cells are differentiated from embryonic mesoderm and neural, retina and epidermal cells are derived from the embryonic ectoderm.

According to preferred embodiments of the present invention, isolating is effected by sorting of cells of the EBs via fluorescence activated cell sorter (FACS).

Methods of isolating EB-derived-differentiated cells via FACS analysis are known in the art. According to one method, EBs are disaggregated using a solution of Trypsin and EDTA (0.025% and 0.01%, respectively), washed with 5% fetal bovine serum (FBS) in phosphate buffered saline (PBS) and incubated for 30 min on ice with fluorescently-labeled antibodies directed against cell surface antigens characteristics to a specific cell lineage.

For example, endothelial cells are isolated by attaching an antibody directed against the platelet endothelial cell adhesion molecule-1 (PECAM1) such as the fluorescently-labeled PECAM1 antibodies (30884×) available from PharMingen (PharMingen, Becton Dickinson Bio Sciences, San Jose, Calif., USA) as described in Levenberg, S. et al., (Endothelial cells derived from human embryonic stem cells. Proc. Natl. Acad. Sci. USA. 2002. 99: 4391-4396). Hematopoietic cells are isolated using fluorescently-labeled antibodies such as CD34-FITC, CD45-PE, CD31-PE, CD38-PE, CD90-FITC, CD117-PE, CD15-FITC, class I-FITC, all of which IgG1 are available from PharMingen, CD133/1-PE (IgG1) (available from Miltenyi Biotec, Auburn, Calif.), and glycophorin A-PE (IgG1), available from Immunotech (Miami, Fla.).

Live cells (i.e., without fixation) are analyzed on a FACScan (Becton Dickinson Bio Sciences) by using propidium iodide to exclude dead cells with either the PC-LYSIS or the CELLQUEST software. It will be appreciated that isolated cells can be further enriched using magnetically-labeled second antibodies and magnetic separation columns (MACS, Miltenyi) as described by Kaufman, D. S. et al., (Hematopoietic colony-forming cells derived from human embryonic stem cells. Proc. Natl. Acad. Sci. USA. 2001, 98: 10716-10721).

Alternatively, isolation of EB-derived differentiated cells is effected by mechanical separation of cells, tissues and/or tissue-like structures contained within the EBs.

For example, beating cardiomyocytes can be isolated from EBs as disclosed in U.S. Pat. Appl. No. 20030022367. Four-day-old EBs of the present invention are transferred to gelatin-coated plates or chamber slides and are allowed to attach and differentiate. Spontaneously contracting cells, which are observed from day 8 of differentiation, are mechanically separated and collected into a 15-mL tube containing low-calcium medium or PBS. Cells are dissociated using Collagenase B digestion for 60-120 minutes at 37° C., depending on the Collagenase activity. Dissociated cells are then resuspended in a differentiation KB medium (85 mM KCl, 30 mM K₂HPO₄, 5 mM MgSO₄, 1 mM EGTA, 5 mM creatine, 20 mM glucose, 2 mM Na₂ATP, 5 mM pyruvate, and 20 mM taurine, buffered to pH 7.2, Maltsev et al., Circ. Res. 75:233, 1994) and incubated at 37° C. for 15-30 min. Following dissociation cells are seeded into chamber slides and cultured in the differentiation medium to generate single cardiomyocytes capable of beating.

According to still additional preferred embodiments of the present invention, isolation of EB-derived-differentiated cells is effected by subjecting the EBs to differentiation factors to thereby induce differentiation of the EBs into lineage specific differentiated cells.

Following is a non-limiting description of a number of procedures and approaches for inducing differentiation of EBs to lineage specific cells.

Neural Precursor Cells

To differentiate the EBs of the present invention into neural precursors, four-day-old EBs are cultured for 5-12 days in tissue culture dishes including DMEM/F-12 medium with 5 mg/ml insulin, 50 mg/ml transferrin, 30 nM selenium chloride, and 5 mg/ml fibronectin (ITSFn medium, Okabe, S. et al., 1996, Mech. Dev. 59: 89-102). The resultant neural precursors can be further transplanted to generate neural cells in vivo (Brustle, O. et al., 1997. In vitro-generated neural precursors participate in mammalian brain development. Proc. Natl. Acad. Sci. USA. 94: 14809-14814). It will be appreciated that prior to their transplantation, the neural precursors are trypsinized and triturated to single-cell suspensions in the presence of 0.1% DNase.

Oligodendrocytes and Myelinate Cells

EBs of the present invention can differentiate to oligodendrocytes and myelinate cells by culturing the cells in modified SATO medium, i.e., DMEM with bovine serum albumin (BSA), pyruvate, progesterone, putrescine, thyroxine, triiodothryonine, insulin, transferrin, sodium selenite, amino acids, neurotrophin 3, ciliary neurotrophic factor and Hepes (Bottenstein, J. E. & Sato, G. H., 1979, Proc. Natl. Acad. Sci. USA 76, 514-517; Raff, M. C., Miller, R. H., & Noble, M., 1983, Nature 303: 390-396]. Briefly, EBs are dissociated using 0.25% Trypsin/EDTA (5 min at 37° C.) and triturated to single cell suspensions. Suspended cells are plated in flasks containing SATO medium supplemented with 5% equine serum and 5% fetal calf serum (FCS). Following 4 days in culture, the flasks are gently shaken to suspend loosely adhering cells (primarily oligodendrocytes), while astrocytes are remained adhering to the flasks and further producing conditioned medium. Primary oligodendrocytes are transferred to new flasks containing SATO medium for additional two days. Following a total of 6 days in culture, oligospheres are either partially dissociated and resuspended in SATO medium for cell transplantation, or completely dissociated and a plated in an oligosphere-conditioned medium which is derived from the previous shaking step [Liu, S. et al., (2000). Embryonic stem cells differentiate into oligodendrocytes and myelinate in culture and after spinal cord transplantation. Proc. Natl. Acad. Sci. USA. 97: 6126-6131].

Mast Cells

For mast cell differentiation, two-week-old EBs of the present invention are transferred to tissue culture dishes including DMEM medium supplemented with 10% FCS, 2 mM L-glutamine, 100 units/ml penicillin, 100 mg/ml streptomycin, 20% (v/v) WEHI-3 cell-conditioned medium and 50 ng/ml recombinant rat stem cell factor (rrSCF, Tsai, M. et al., 2000. In vivo immunological function of mast cells derived from embryonic stem cells: An approach for the rapid analysis of even embryonic lethal mutations in adult mice in vivo. Proc Natl Acad Sci USA. 97: 9186-9190). Cultures are expanded weekly by transferring the cells to new flasks and replacing half of the culture medium.

Hemato-Lymphoid Cells

To generate hemato-lymphoid cells from the EBs of the present invention, 2-3 days-old EBs are transferred to gas-permeable culture dishes in the presence of 7.5% CO₂ and 5% O₂ using an incubator with adjustable oxygen content. Following 15 days of differentiation, cells are harvested and dissociated by gentle digestion with Collagenase (0.1 unit/mg) and Dispase (0.8 unit/mg), both are available from F. Hoffman-La Roche Ltd, Basel, Switzerland. CD45-positive cells are isolated using anti-CD45 monoclonal antibody (mAb) M1/9.3.4.HL.2 and paramagnetic microbeads (Miltenyi) conjugated to goat anti-rat immunoglobulin as described in Potocnik, A. J. et al., (Immunology Hemato-lymphoid in vivo reconstitution potential of subpopulations derived from in vitro differentiated embryonic stem cells. Proc. Natl. Acad. Sci. USA. 1997, 94: 10295-10300). The isolated CD45-positive cells can be further enriched using a single passage over a MACS column (Miltenyi).

It will be appreciated that the culturing conditions suitable for the differentiation and expansion of the isolated lineage specific cells include various tissue culture medium, growth factors, antibiotic, amino acids and the like and it is within the capability of one skilled in the art to determine which conditions should be applied in order to expand and differentiate particular cell types and/or cell lineages.

In addition to the lineage-specific primary cultures, EBs of the present invention can be used to generate lineage-specific cell lines which are capable of unlimited expansion in culture.

Cell lines of the present invention can be produced by immortalizing the EB-derived cells by methods known in the art, including, for example, expressing a telomerase gene in the cells (Wei, W. et al., 2003. Abolition of Cyclin-Dependent Kinase Inhibitor p16Ink4a and p21Cip1/Waf1 Functions Permits Ras-Induced Anchorage-Independent Growth in Telomerase-Immortalized Human Fibroblasts. Mol Cell Biol. 23: 2859-2870) or co-culturing the cells with NIH 3T3 hph-HOX11 retroviral producer cells (Hawley, R. G. et al., 1994. The HOX11 homeobox-containing gene of human leukemia immortalizes murine hematopoietic precursors. Oncogene 9: 1-12).

To express the telomerase gene in mammalian cells, a polynucleotide encoding telomerase is ligated into an expression vector under the control of a promoter suitable for mammalian cell expression.

The polynucleotide of the present invention is a genomic or complementary polynucleotide sequence which encodes the telomerase gene such as for example, homo sapiens telomerase (GenBank Accession No: NM_{—}003219) or mouse telomerase (GenBank Accession Nos: AF051911, AF073311).

As is mentioned hereinabove, to enable mammalian cell expression, the expression vector of the present invention includes a promoter sequence for directing transcription of the polynucleotide sequence in a mammalian cell in a constitutive or inducible manner.

Constitutive promoters suitable for use with the present invention are promoter sequences which are active under most environmental conditions and most types of cells such as the cytomegalovirus (CMV) and Rous sarcoma virus (RSV). Inducible promoters suitable for use with the present invention include for example the hypoxia-inducible factor 1 (HIF-1) promoter (Rapisarda, A. et al., 2002. Cancer Res. 62: 4316-24) and the tetracycline-inducible promoter (Srour, M. A. et al., 2003. Thromb. Haemost. 90: 398-405).

The expression vector of the present invention includes additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for the present invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation of the gene of interest (e.g., telomerase). Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for the present invention include those derived from SV40.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of the present invention can further include additional polynucleotide sequences that allow, for example, the translation of several proteins from a single mRNA such as an internal ribosome entry site (IRES) and sequences for genomic integration of the promoter-chimeric polypeptide.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by the present invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I).

Recombinant viral vectors are useful for in vivo expression of the gene of interest (e.g., telomerase) since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of the present invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Since the lineage-specific cells of the present invention are developed by differentiation processes similar to those naturally occurring in the human embryo they can be further used for human cell-based therapy and tissue regeneration.

Thus, according to another aspect of the present invention there is provided a method of treating a disorder requiring cell replacement therapy. The method according to this aspect of the present invention is effected by administering the expanded and/or differentiated lineage-specific cells of the present invention to an individual in need thereof, thereby treating the disorder requiring cell replacement therapy.

As used herein “treating a disorder requiring cell replacement therapy” refers to treating an individual suffering from a disorder such as a neurological disorder, a muscular disorder, a cardiovascular disorder, an hematological disorder, a skin disorder, a liver disorder, and the like that require cell replacement.

The phrase “treating” refers to inhibiting or arresting the development of a disease, disorder or condition and/or causing the reduction, remission, or regression of a disease, disorder or condition in an individual suffering from, or diagnosed with, the disease, disorder or condition. Those of skill in the art will be aware of various methodologies and assays which can be used to assess the development of a disease, disorder or condition, and similarly, various methodologies and assays which can be used to assess the reduction, remission or regression of a disease, disorder or condition.

As used herein, “administering” refers to means for providing the expanded and/or differentiated lineage specific cells to an individual, using any suitable route, e.g., oral, sublingual intravenous, subcutaneous, transcutaneous, intramuscular, intracutaneous, intrathecal, intra peritoneal, intra spleenic, intra hepatic, intra pancreatic, intra cardiac, epidural, intraoccular, intracranial, inhalation, rectal, vaginal, and the like administration.

Differentiated stem cells of lineage specific origin can be utilized in treating various disorders. For example, oligodendrocyte precursors can be used to treat myelin disorders (Repair of myelin disease: Strategies and progress in animal models. Molecular Medicine Today. 1997. pp. 554-561), chondrocytes or mesenchymal cells can be used in treatment of bone and cartilage defects (U.S. Pat. No. 4,642,120) and cells of the epithelial lineage can be used in skin regeneration of a wound or burn (U.S. Pat. No. 5,716,411).

For certain disorders, such as genetic disorders in which a specific gene product is missing [e.g., lack of the CFTR gene-product in cystic fibrosis patients (Davies J C, 2002. New therapeutic approaches for cystic fibrosis lung disease. J. R. Soc. Med. 95 Suppl 41:58-67)], ESC-derived cells are preferably manipulated to over-express the mutated gene prior to their administration to the individual. It will be appreciated that for other disorders, the ESC-derived cells should be manipulated to exclude certain genes.

Over-expression or exclusion of genes can be effected using knock-in and/or knock-out constructs.

Knock-out and/or knock-in constructs can be used in somatic and/or germ cells gene therapy to destroy activity of a defective allele, gain of function (e.g., dominant) allele, or to replace the lack of activity of a silent allele in an individual, thereby down or up-regulating activity of specific genes, as required. Further detail relating to the construction and use of knockout and knock-in constructs can be found in Fukushige, S, and Ikeda, J. E.: Trapping of mammalian promoters by Cre-lox site-specific recombination. DNA Res 3 (1996) 73-50; Bedell, M. A., Jerkins, N. A. and Copeland, N. G.: Mouse models of human disease. Part I: Techniques and resources for genetic analysis in mice. Genes and Development 11 (1997) 1-11; Bermingham, J. J., Scherer, S. S., O'Connell, S., Arroyo, E., Kalla, K. A., Powell, F. L. and Rosenfeld, M. G.: Tst-1/Oct-6/SCIP regulates a unique step in peripheral myelination and is required for normal respiration. Genes Dev 10 (1996) 1751-62, which are incorporated herein by reference.

In addition to cell replacement therapy, the lineage specific cells of the present invention can also be utilized to prepare a cDNA library. mRNA is prepared by standard techniques from the lineage specific cells and is further reverse transcribed to form cDNA. The cDNA preparation can be subtracted with nucleotides from embryonic fibroblasts and other cells of undesired specificity, to produce a subtracted cDNA library by techniques known in the art.

The lineage specific cells of the present invention can be used to screen for factors (such as small molecule drugs, peptides, polynucleotides, and the like) or conditions (such as culture conditions or manipulation) that affect the differentiation of lineage precursor to terminally differentiated cells. For example, growth affecting substances, toxins or potential differentiation factors can be tested by their addition to the culture medium.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Experimental Procedures

hESC culture—Undifferentiated hESCs (H9.2 and H13) were used. The cells were grown on an inactivated mouse embryonic feeder layer (MEF), as previously described (Amit et al, 2000). hESCs were separated, using type IV collagenase, resulting in small aggregates. For conventional static hEB formation, hESCs were removed from the feeder layers using either 1 mg/ml type IV collagenase (Gibco Invitrogen Co., San Diego Calif., USA) or 5 mM EDTA in PBS, then cultured in suspension in 50 mm non-adherent Petri dishes (Ein-Shemer, Israel). The hEBs were grown in a medium consisting of 80% KO-DMEM (Gibco Invitrogen Co., San Diego Calif., USA), supplemented with 20% defined fetal bovine serum (FBSd; HyClone), 1 mM L-glutamine, and 1% non-essential amino acid stock (all from Gibco-BRL). Dynamic formation of hEBs was effected using a slow turning lateral vessel (STLV), as previously described (Gerecht-Nir et al, 2004).

Scaffold production—3D alginate scaffolds were generated as previously described (Shapiro and Cohen, 1997; Zmora et al, 2002). Briefly, 3-D alginate scaffolds were prepared from a pharmaceutical-grade alginate, Protanal LF 5/60 or LF120 (FMC Biopolymers, Drammen, Norway), which has a high guluronic acid (G) content (65-75%) and solution viscosity (1% w/v, 25° C.) of 50 and 200 cP, respectively. Alginate scaffold was generated by a 4-step process; (i) preparation of sodium alginate stock solutions at concentrations of 1-3% (w/v); (ii) cross-linking of the alginate by drop-wise adding of the bivalent cross-linker, e.g., calcium gluconate; (iii) freezing the cross-linked alginate using a homogenous cool (−20° C.) environment; and (iv) lyophilization to produce a sponge-like scaffold. The scaffolds were sterilized, using the ethylene oxide gas apparatus and stored in laminated bags at room temperature until use. Scaffolds thus generated had the following dimensions: 5 mm×2 mm (d×h). It will be appreciated that such thickness (h) was allowed based on preliminary findings indicating that mass transfer limitations in such scaffolds are not as limiting. The scaffolds were featured by 90% porosity having a pore size ranging from 50-200 μm in diameter (Shapiro and Cohen, 1997; Zmora et al, 2002).

Cell seeding in scaffold and cultivation—Dynamic cell seeding onto the scaffold was effected as described by Dar et al (2002). Undifferentiated hESCs were collected, counted and concentrated according to the desired seeding cell number (see Example 2 of the Examples section) into 10-20 μl of hEB medium. The small volume cell-suspension was applied to the scaffolds, followed by a centrifugation stage of 1500 rpm for 3 minutes and the addition of 200 μl of EB medium. After 12-24 hours, the scaffolds were placed in 12-well plates (Nunc, Roskilde, Denmark) with 1 ml of fresh medium. Residual un-entrapped cells were counted, using trypan blue dye or an XTT assay (see below). The results are expressed as mean±SD.

hEB size, histology and immunohistochemistry—To analyze the number and size of the scaffold-borne hEBs, the alginate scaffolds were dissolved in PBS in which the phosphate ions are used as chelators for calcium ions. The released hEBs were transferred into culture dishes and analyzed using inverted-light microscopy (IX50 inverted system microscopy, Olympus Optical Co., LTD. Tokyo, Japan). The counts of total hEBs from 5 fields of ×100 magnification were averaged. For size analysis, the diameter average was calculated by measuring the large and small diagonals of 5 representative hEBs from each field. The results of measurements from two independent experiments are presented. The number of scaffolds used in each experiment is presented in the results.

For histological analyses, scaffolds seeded with cells were fixed in 10% neutral-buffered formalin for one hour at room temperature, dehydrated in graduated alcohol (70-100%), and embedded in paraffin for routine histology. 6-8 μm sections were stained with hematoxylin/eosin. Immunostaining was performed with a Dako LSAB®+ staining kit with specific anti-human CD34, anti-human α feto-protein (AFP) (all from Dako, Denmark), anti-human Nestin (R&D Systems, Minneapolis Minn., USA), and anti-human stage-specific embryonic antigen 4 (SSEA4, kindly provided by Prof. P. Andrews, University of Sheffield, UK). Mouse IgG isotype-matching (R&D Systems, Minneapolis Minn., USA) or secondary antibody alone (from Dako LSAB®+ staining kit), were used as a control. For quantification, at least 20 hEBs per scaffold (n=5) of three different experiments were scored for positive CD34+ voids. Vascularization degree was calculated as the number of positive voids per hEB. The results were expressed as mean±SD.

Viability assay—Viable cell concentration was determined by the XTT Kit (Sigma, St Louis Mo., USA), according to manufacturer's instructions. Briefly, cell-seeded scaffolds were incubated for 4 hrs with EB differentiation medium containing 20% (v/v) XTT solution. 150 μl of the medium were removed, placed in a 96-plate well and read by a microplate reader at 450 nm. Cell concentration was determined according to the standard curve of known cell concentrations as previously described, Gerecht-Nir et al. (2004).

Scanning electron microscopy—Scaffolds were fixed for 1 hr in 3% glutaraldehyde in 0.1 M sodium cacodylate, dehydrated in graduated alcohol (70-100%) and dried. The samples were coated with gold and examined using Philips XL30 (FEI company, Eindhoven, The Netherlands).

### Example 1

### Generation of Alginate Scaffolds

The present invention hypothesized that the confined environment of the pore structure in the scaffold would enable the formation of a homogeneous population of hEBs minimizing hEB agglomeration and resulting in efficient cell proliferation and differentiation. To test this, alginate scaffolds were generated.

Results

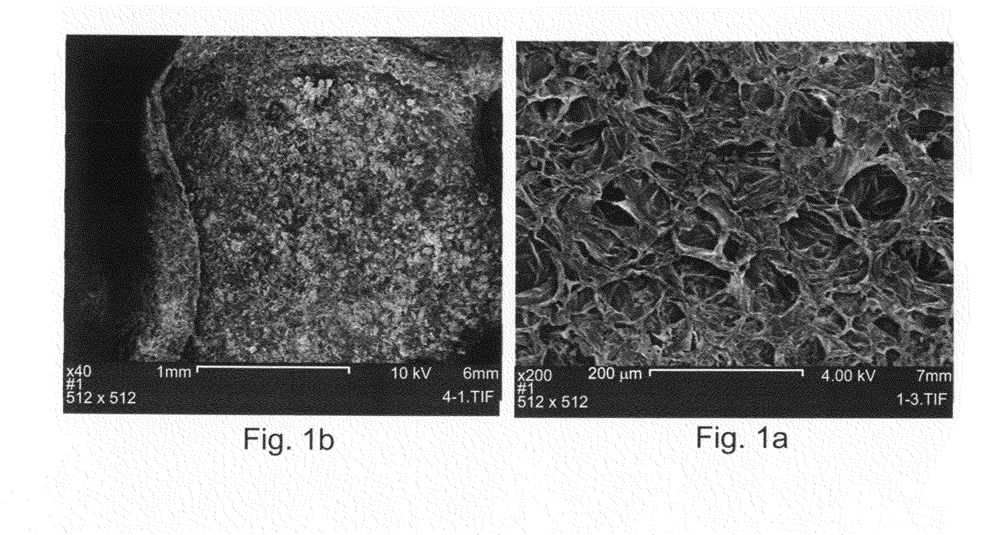

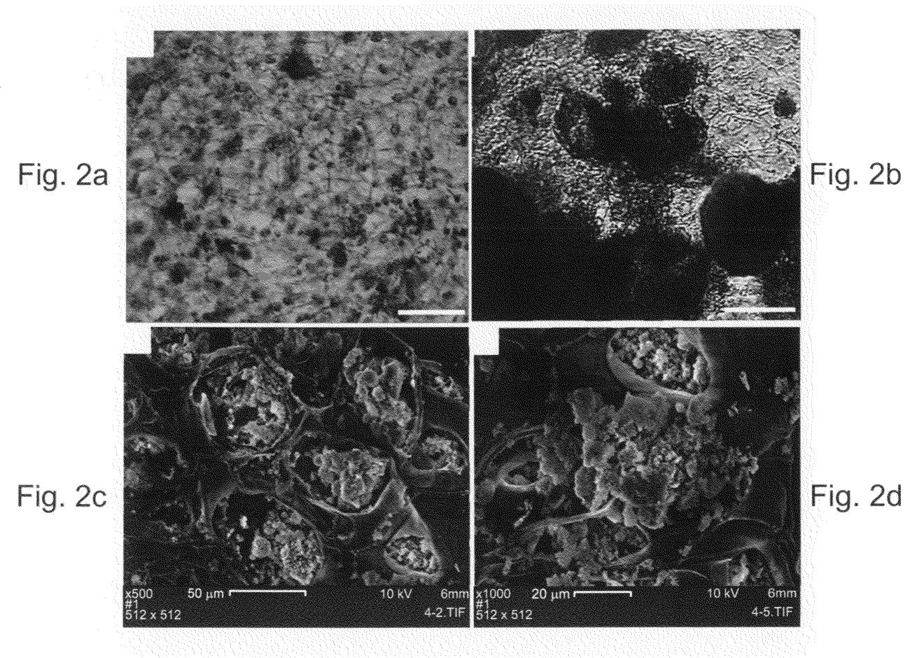

Alginate scaffolds were fabricated from pure alginate, LF5/60 or LF120, which has a high guluronic acid content. The scaffolds were characterized by 90% porosity, interconnecting pore structure and homogenous isotropic round pores with an average pore diameter of 100 μm (Zmora et al, 2002; FIG. 1a). In their dry state, the scaffolds from the LF5/60 alginate demonstrated lower values of elastic modulus (500 vs. 1,136±264 kPa for the LF120 scaffold) and in culture medium, they degraded at a faster rates compared to those made of the LF120 alginate (Zmora et al, 2002). The hydrophilic nature of the alginate material enabled the rapid wetting of the scaffolds by the culture medium, resulting in efficient cell seeding. Seeding the scaffolds with suitable cell concentration (see Example 2, below), resulted in their even distribution all over the scaffold pores (FIG. 1b).

### Example 2

### Efficiency of Human EB Formation within Alginate Scaffolds

The capability of the 3-D porous alginate scaffolds to efficiently entrap the seeded hESCs and support the formation of hEBs was examined.

Results

To test the ability of alginate scaffolds to support EB formation, undifferentiated hESCs were removed from their feeder layer and dynamically seeded at different cell concentrations onto the alginate scaffolds prepared from either LF5/60 or LF120 alginate with the following dimensions: 5 mm diameter and 1-2 mm thickness. Three initial cell-seeding concentrations were investigated: (1) high density cell seeding, ranging from 0.8-1×10⁶ cells per scaffold; (2) medium density cell seeding, ranging from 0.4-0.7×10⁶ cells per scaffold; and (3) low density cell seeding, ranging from 0.1-0.25×10⁶ cells per scaffold. Moderate centrifugal force was applied during hESC seeding resulting in a uniform cell distribution throughout the alginate scaffolds as previously described with cardiomyocytes cell seeding (Dar et al, 2002, see FIG. 1b). As expected, adherence of the cells seeded onto the scaffolds and the number and size of the resultant hEBs depended on the initial cell seeding concentration and the type of scaffold used. Specifically, seeding hESCs onto the LF5/60 scaffolds (n=20) resulted in the adherence of 62.34±5.16% of the initial seeded cells within 24 hrs, irrespective of the initial cell seeding concentration. The cells, which adhered to the scaffolds, aggregated to form hEBs within 48 hours (FIG. 2a). Although hEBs formation was not affected by the initial seeding concentration, the density of forming hEBs increased concomitantly with the increase in initial cell seeding concentration, as occurs in conventional hEB formation using Petri-dishes. With time, the LF5/60 scaffold-borne hEBs grew in size. hEB growth has lead to the rupture of the scaffolds due to their relatively poor mechanical strength and fast degradation, and the hEBs leaked out to the medium from around day 15 and on in culture (FIG. 2b).

When using the LF120 alginate scaffolds, the initial cell seeding concentration affected the degree of cell adherence to the scaffold as well as the extent of hEBs formation. However, when using too a low concentration of cells efficient adherence was achieved but no EBs were formed. Thus, almost all cells adhered (92.7±3.02%) when seeding the scaffolds (n=20) with low-cell concentration suspensions, but no hEBs were formed under these conditions. When seeding with medium-cell concentrations (n=12), 78.44±4.62% of the cells adhered to the scaffolds and formed hEBs within 48 hours. High cell seeding concentration (n=10) resulted in a relatively lower adherence percentage (61.05±1.2%) and formation of hEBs was observed to occur in the entire scaffold pores. Scanning electron micrographs of the hESC-seeded scaffolds following 1 month cultivation revealed that hEBs occupied the entire pore volume (FIG. 2c), sometime extending outside the pores (FIG. 2d). Seeding the scaffolds with either high or medium cell concentrations yielded hEBs with a similar size, which with time, grew in size, eventually reaching a diameter ranging from 250 to 900 μm after one month on culture. Apparently, scaffold degradation with time and the increase in scaffold pore size enabled the growth of hEBs beyond the initial scaffold pore size of 100 μm in diameter.

The LF120 alginate scaffold were employed for further analysis, using medium-cell seeding concentrations. Under these conditions, hEBs were formed mainly within the scaffold pores and were distributed evenly over the entire scaffold volume. It seems that the relatively small pore size, along with the hydrophilic nature of the alginate scaffold, allowed the generation of hEBs with moderate distribution.

Similarly to the EBs formed in the rotary STLV bioreactor (Gerecht-Nir et al, 2004), the scaffold-borne hEBs, were small in size (ranging from 250 to 900 μm following one month of culture, as mentioned above), with a round shape displaying minimal agglomeration. However, unlike the hEBs formed in the bioreactors, the scaffold-borne hEBs were a less homogeneous population in terms of particle size, probably reflecting the variability of scaffold pore size. Overall, it appears that the environments of rotating culture and 3D-scaffolds induce round hEBs while minimizing their agglomeration compared to hEBs formed in Petri dishes.

### Example 3

### Cell Viability in the Scaffold System

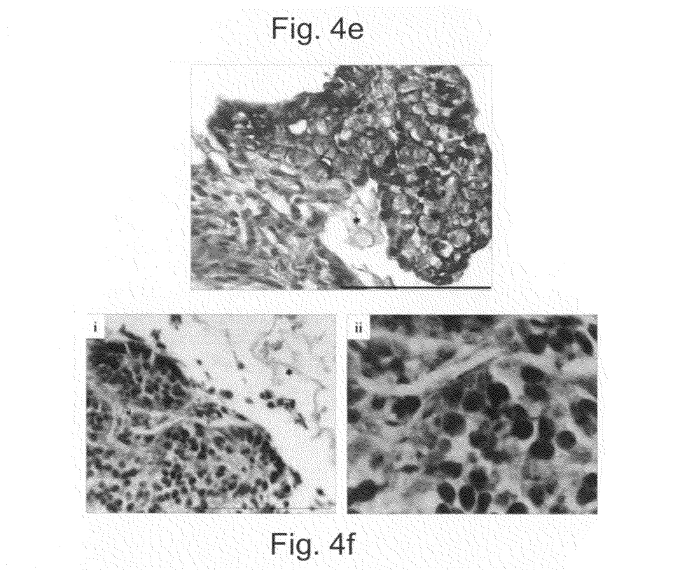

The number of viable cells in the time-course of hEB formation and culture in alginate scaffolds was determined by the XTT viability assay and compared to previously reported rates of EB formation in a static culture using Petri dishes and in a dynamic culture using the STLV bioreactor (Gerecht-Nir et al, 2004). Examination of the proliferation rate during hEB formation within alginate scaffolds revealed a two-fold increase in viable cell concentration within the first week of culture, as previously reported for static culture (Gerecht-Nir et al, 2004). Starting from the second week in culture, an increase in viable cell concentration in the scaffold system was observed, exceeding that of the static cultures by two-fold (FIG. 3).

### Example 4

### Morphology and Differentiation of Cells in the Scaffold System

hEBs formed within the alginate scaffolds grew within the scaffold pores, proliferated and differentiated. Examination of their general morphology in histology sections performed on day 30 in culture revealed the formation of fairly round hEBs with an internal overall appearance, similar to those formed within the STLV bioreactor. The extent of hEB aggregation in the scaffold was minimal as in the bioreactor while in the static Petri dishes the hEBs aggregated with time.

Different cell types and tissue-like structures such as epithelial sheets and tubes, connective tissues and various voids could be observed in the scaffold-borne hEBs which remained entrapped inside the scaffold pores, as well as in those that burst out from the scaffold (FIGS. 4b-d).

The presence of undifferentiated cells within the scaffold-borne hEBs was examined by SSEA4, a known specific marker for primate cells of the inner cell mass stage (Thomson et al, 1998). 1-, 2- and 4-week-old scaffold-borne hEBs were examined while Petri dish-borne hEBs served as a control. In both culture systems, after one week of differentiation, no SSEA4+ cells could be detected. Furthermore, positive immuno-labeling of representative tissues of the three germ layers demonstrated that the scaffold-borne hEBs have the capability of differentiating into representatives of the three germ layers: ectoderm, endoderm (FIGS. 4e-f), and mesoderm (see in Example 5 hereinbelow). Generally, it seems that the confining environment of the scaffolds does not interrupt the initial differentiation process, but rather controls the morphology and size of hEBs.

### Example 5

### Vasculogenesis within the Scaffold-Borne hEBS

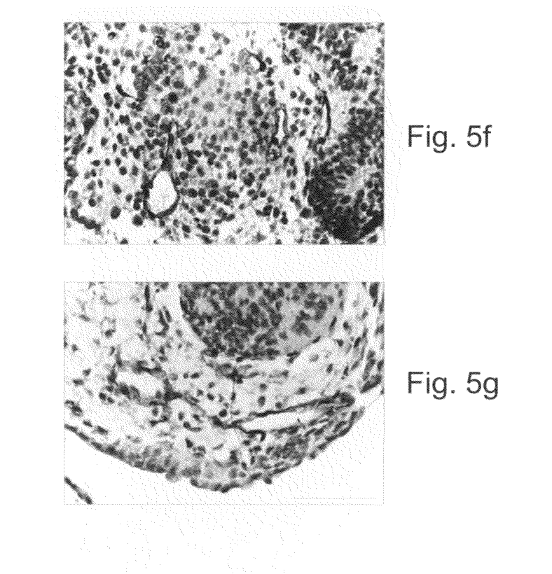

Histological sections of the hEBs formed within the scaffolds revealed an adjacent formation of voids and tube-like structures. These voids were mainly located wherein the cells surrounding the voids were lining the scaffold wall or around several scaffold pores (FIGS. 5a-b). Immuno-labeling showed that the majority of these structures were formed by CD34+ cells (FIG. 5c) and that the CD34+ voids were mostly arranged along the scaffold solid matrix, resulting in a relatively large and complex structure (FIGS. 5d-e) compared to those formed in other systems (FIGS. 5f-g). Furthermore, quantification of the CD34+ cells reveals 3.3±1.02 voids per scaffold-borne hEBs compared to 1.89±0.53 voids per hEBs formed in the Petri dishes and 1.72±0.76 voids per hEBs formed in bioreactors. Thus, the alginate 3D-scaffold environment induces vessel formation to a larger extent as compared to the unconfined environments of the Petri dish and STLV bioreactor.

The enhanced vasculogenesis process in the scaffold-borne hEBs compared to that in the static or rotating culture systems may be explained by the unique environment provided by the porous scaffold matrix. On one hand, the scaffold provides a solid matrix along which the cells can adhere and interact with each other and with the solid matrix and on the other hand, the environment of the medium-filled pores allow the cells to aggregate thereby mimicking suspension cultures. The simultaneous occurrences of both processes may lead to the highly vascularized hEBs. Furthermore, it is possible that cell adherence in the alginate scaffolds induces different cell signaling processes which favor vasculogenesis in the forming hEBs. Such culture conditions are not available in the static Petri dishes or in the rotating bioreactors.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED

### Other References are Fully Cited in the Application

Amit M, Carpenter M K, Inokuma M S, Chiu C P, Harris C P, Waknitz M A, Itskovitz-Eldor J, Thomson J A. 2000. Clonally derived human embryonic stem cell lines maintain pluripotency and proliferative potential for prolonged periods of culture. Dev Biol 227:271-278.
Chen S S, Revoltella R P, Papini S, Michelini M, Fitzgerald W, Zimmerberg J, Margolis L. 2003. Multilineage differentiation of rhesus monkey embryonic stem cells in three-dimensional culture systems. Stem Cells 21:281-295.
Czyz J, Wobus A. 2001. Embryonic stem cell differentiation: the role of extracellular factors. Differentiation 68:167-174.
Dar A, Shachar M, Leor J, Cohen S. 2002. Optimization of cardiac cell seeding and distribution in 3D porous alginate scaffolds. Biotechnol Bioeng 80:305-312.
Gerecht-Nir S, Cohen S, Itskovitz-Eldor J. 2004. Bioreactor cultivation enhances the efficiency of human embryoid body (hEB) formation and differentiation. Biotechnol Bioeng. In press.
Gerecht-Nir S, Ziskind A, Cohen S, Itskovitz-Eldor J. 2003. Human embryonic stem cells as an in vitro model for human vascular development and the induction of vascular differentiation. Lab Invest 83:1811-1820.
Glicklis R, Shapiro L, Agbaria R, Merchuk J C, Cohen S. 2000. Hepatocyte behavior within three-dimensional porous alginate scaffolds. Biotechnol Bioeng 67:344-353.
Itskovitz-Eldor J, Schuldiner M, Karsenti D, Eden A, Yanuka O, Amit M, Soreq H, Benvenisty N. 2000. Differentiation of human embryonic stem cells into embryoid bodies compromising the three embryonic germ layers. Mol Med 6:88-95.
Leor J, Aboulafia-Etzion S, Dar A, Shapiro L, Barbash I, Battler A, Granot Y, Cohen S. 2000. Bioengineered cardiac grafts: A new approach to repair the infarcted myocardium. Circulation 102:II-56-61.
Levenberg S, Golub J S, Amit M, Itskovits-Eldor J, Langer R. 2002. Endothelial cells derived from human embryonic stem cells. Proc Natl Acad Sci USA 99:4391-4396.
Levenberg S, Huang N F, Lavik E, Rogers A B, Itskovitz-Eldor J, Langer R. 2003. Differentiation of human embryonic stem cells on three-dimensional polymer scaffolds. Proc Natl Acad Sci USA 100:12741-12746.
Shapiro L, Cohen S. 1997. Novel alginate sponges for cell culture and transplantation. Biomaterials 18:583-590.
Thomson J A, Itskovitz-Eldor J, Shapiro S S, Waknitz M A, Swiergiel J J, Marshall V S, Jones J M. 1998. Embryonic stem cell lines derived from human blastocysts. Science 282:1145-1147.
Zmora S, Glicklis R, Cohen S. 2002. Tailoring the pore architecture in 3-D alginate scaffolds by controlling the freezing regime during fabrication. Biomaterials 23:4087-4094.

1. A method of generating embryoid bodies comprising culturing undifferentiated embryonic stem cells on a three dimensional scaffold under conditions suitable for formation of embryoid bodies, thereby generating the embryoid bodies.

2. A method of generating expanded and/or differentiated cells from embryonic stem cells comprising:
(a) culturing undifferentiated embryonic stem cells on a three dimensional scaffold under conditions suitable for formation of embryoid bodies to thereby obtain embryoid bodies;
(b) isolating lineage specific cells from said embryoid bodies; and
(c) culturing said lineage specific cells under culturing conditions selected suitable for the expansion and/or differentiation of said lineage specific cells to thereby obtain expanded and/or differentiated lineage-specific cells.

3. Use of scaffold-borne embryoid bodies or lineage specific cells isolated therefrom as a pharmaceutical.

4. Use of scaffold-borne embryoid bodies or lineage specific cells isolated therefrom for the manufacture of a medicament useful for treating a disorder requiring cell replacement therapy.

5. The use or method of claim 1, wherein said three dimensional scaffold is a porous scaffold.

6. The use or method of claim 5, wherein said porous scaffold is an alginate scaffold.

7. The use or method of claim 6, wherein said alginate scaffold is an LF120 alginate scaffold or an LF5/60 alginate scaffold.

8. The use or method of claim 5 wherein said porous scaffold is composed of a synthetic polymer.

9. The use or method of claim 5, wherein said porous scaffold is composed of a natural polymer.

10. The use or method of claim 8, wherein said synthetic polymer is selected from the group consisting of a poly(hydroxy) acid, polyanhydride, poly(ortho)ester and polyurethane.

11. The use or method of claim 10, wherein said poly(hydroxy) acid is selected from the group consisting of PLA, PLGA, PGA and PEG containing co-polymers thereof.

12. The use or method of claim 9, wherein said natural polymer is selected from the group consisting of a polysaccharide and a polypeptide.

13. The use or method of claim 12, wherein said polysaccharide is selected from the group consisting of alginate, chitosan and hyaluronic acid.

14. The use or method of claim 5, wherein an average pore size of said porous scaffold is in a range between 10 to 900 μm in diameter.

15. The use or method of claim 5, wherein an average distance between pores of said porous scaffold is in a range between 5 to 500 μm.

16. The use or method of claim 5, wherein an average porosity of said porous scaffold is at least 70%.

17. The method of claim 1, wherein said culturing is effected over a period of 1-35 days.

18. The method of claim 1, wherein said culturing is effected for 30 days.

19. The use or method of claim 1, wherein at least 90% of the embryoid bodies are within a diameter size range of 400-800 μm.

20. The use or method of claim 1, wherein at least 85% of the embryoid bodies are devoid of necrotic centers.

21. The method of claim 1, wherein conditions suitable for formation of embryoid bodies include a culture medium containing serum.

22. The method of claim 1, wherein said culture medium includes 80% KO-DMEM, 20% serum, 0.5% Penicillin-Streptomycin, 1 mM L-glutamine, 0.1 mM β-mercaptoethanol and 1% non-essential amino acid stock.

23. The method of claim 2, wherein step (b) is effected by sorting of cells contained within said embryoid bodies via fluorescence activated cell sorter.

24. The method of claim 2, wherein step (b) is effected by a mechanical separation of cells, tissues and/or tissue-like structures contained within said embryoid bodies.

25. The method of claim 2, wherein said isolating lineage specific cells is effected by subjecting said embryoid bodies to differentiation factors to thereby induce differentiation of said embryoid bodies into lineage specific differentiated cells.
